(19) United States
(12) Patent Application Publication
(10) Pub. No.: US 2011/0106099 A1
(43) Pub. Date: May 5, 2011

(54) LEAD EXTRACTION DEVICE

(75) Inventors: Niall F. Duffy, Tuam (IE); James S. Smedley, Tuam (IE)

(73) Assignee: Medtronic, Inc., Minneapolis, MN (US)

(21) Appl. No.: 12/608,687

(22) Filed: Oct. 29, 2009

(51) Int. Cl. A61B 17/00

(52) U.S. Cl. 606/129

(57) ABSTRACT

The disclosure pertains to a lead extraction device which utilizes a chamfered coring tip to separate an implanted object, such as a pacemaker lead, from fibrous tissue and thereby permit the implanted object to be extracted from a body. The lead extraction device features an elongate body having a lead gripping mechanism. The gripping mechanism grips a lead to prevent kinking or simultaneous rotation of the lead with the lead extraction device that may cause tissue damage. The inner lumen of lead extraction device is preferably dimensioned so that a lead will fit within. The lead extraction device is thereby tracked over the lead. The chamfered coring tip on the distal end of the lead separates the lead from fibrous tissue. Through the disclosed lead extraction devices of the present disclosure, the lead may be separated along its length, as well as separated at its distal end from fibrous tissue, thereby permitting the lead to be readily extracted from the body.

### TECHNICAL FIELD

The present disclosure generally relates to an apparatus and method for removal of an implanted object from a patient's body. More specifically and without limitation, this disclosure relates to removal of leads from a patient's heart and the venous paths thereto.

### BACKGROUND

Many implantable medical devices such as pacemakers, defibrillators and neural stimulators deliver electrical therapy to tissue and sense various physiological parameters via medical leads. Such leads typically include an insulated and elongated flexible lead body surrounding one or more conductors extending between the proximal and distal ends of the lead. The conductors are typically coupled to one or more electrodes disposed at a distal end of the lead for positioning in a chamber or portion of the heart such as the right atrium, right ventricle, the right atrial appendage or the coronary sinus.

Lead electrodes are placed in contact with myocardial tissue by passage of the lead through a vein such as the subclavian vein or one of its tributaries. The tip of the lead is typically held in place by trabeculae present in the myocardial tissue. A fixation mechanism is often provided at the distal end of the lead to enhance the chronic stability of lead positioning and electrode placement. Among the many available types of leads are those having either “active fixation” or “passive fixation” mechanisms.

Known passive fixation mechanisms include flexible tines, wedges, or finger-like projections that extend radially outward and usually are molded from and/or are integral with the insulating sheath of the lead. These tines or protrusions allow surrounding growth of tissue and scar in chronically implanted leads to fix the electrode tip in position in the heart and prevent dislodgment of the tip during the life of the lead.

Known types of active fixation mechanisms for cardiac leads include “screw-in” tips in which a sharpened helical or “corkscrew” needle is provided at the distal end of the lead for engaging myocardial tissue. In some screw-in leads, the helical needle is capable of being screwed in to endocardial tissue by means of a slotted-tip or screwdriver-tip stylet which is inserted into the lead during the implantation process and rotated at its proximal end to secure the distal end in place. Examples of screw-in leads are described in U.S. Pat. No. 4,886,074 to Bisping, U.S. Pat. No. 4,217,913 to Dutcher, U.S. Pat. No. 4,967,766 to Bradshaw, and in U.S. Pat. No. 5,002,067 to Berthelsen et al.

Due to a variety of reasons, such as the desire to upgrade to newer technology, there may be a need to explant/remove a lead. Leaving an unused lead in the body may be an alternative. However, in some cases the unused lead may be associated with patient discomfort or a likelihood that the lead may present a likelihood of infection. Moreover, the presence of unused leads in a venous pathway or inside the heart may cause considerable difficulty in the positioning and attachment of new endocardial leads in the heart.

Various techniques have been proposed for removal of implanted leads. Removal of an inoperative lead sometimes can be accomplished by applying tractional force and rotation to the proximal end of the lead. This method is most effective, however, when done prior to fixation of the lead tip in the trabeculae by fibrous tissue formation. In cases where the lead tip has become attached by fibrous tissue to the myocardial wall, removal of the lead presents additional complexity because of the risk of myocardial wall damage. The application of pulling force upon the proximal end is that the flexible sheath of the lead body can stretch and possibly tear under the applied tractional force. The flexible body of the lead presents additional challenges that further complicate an inherently complicated extraction process.

To address the foregoing problems, various tools and methods for lead removal have been proposed. Examples of such tools include cutting catheters such as that discussed in U.S. Pat. No. 4,576,162 to McCorkle, entitled “Apparatus and Method for Separation of Scar Tissue in Venous Pathway,” incorporated herein by reference in its entirety. Others have proposed the use of a lead extraction device that utilizes laser light to separate an implanted lead from fibrous scar tissue such as the device disclosed in U.S. Pat. No. 5,769,858 to Pearson et al., entitled “Locking Stylet for Extracting Implantable Lead or Catheter,” incorporated herein by reference in its entirety. However, despite the various proposals and techniques, there remains an abundance of challenges to removing implanted leads.

### SUMMARY

In view of the foregoing considerations, the present disclosure is directed to apparatus and methods for the extraction of elongate leads. More particularly, the disclosure is directed to a tubular lead extraction device having a manipulable body that permits the device to track over an implanted lead and separate the lead from fibrous tissue surrounding the lead.

In an embodiment, the lead extraction device may include a shaft coupled to an outer tube. An engagement mechanism may be disposed within the outer tube. The engagement mechanism may have a first portion having a gripping mechanism that selectively engages the lead and a second portion that threadedly engages with the outer tube. A coring tip is coupled to a distal end of the outer tube for separating or cutting-away at the tissue surrounding the lead tip.

In one embodiment, the gripping mechanism is a threaded portion that engages a coupling pin that is coupled to the outer tube. The gripping mechanism anchors the lead extraction device onto the lead to inhibit movement of the lead as the tool separates the lead from tissue. In addition to increasing the separation efficiency of the device, the gripping mechanism also imparts minimal trauma to the myocardium by inhibiting movement of the lead.

### BRIEF DESCRIPTION OF THE DRAWINGS

The following drawings are illustrative of particular embodiments of the present disclosure and therefore do not limit the scope of the disclosure. The drawings (not to scale) are intended for use in conjunction with the explanations in the following detailed description, wherein similar elements are designated by identical reference numerals. Moreover, the specific location of the various features is merely exemplary unless noted otherwise.

FIG. 1 depicts a lead placed in the venous system and having its distal tip located in the heart.

FIG. 2 illustrates a side sectional view of an exemplary lead extraction device.

FIG. 3 is a sectional view of the lead extraction device shown in FIG. 2 taken along the lines 3-3.

FIG. 4 depicts the coupling of a distal portion of the lead extraction device with an implanted lead as it would be used to remove a lead.

FIG. 5 is a side sectional view of the lead extraction device showing the device in a partially-extended configuration.

FIG. 6 is a side sectional view of the lead extraction device showing the device in a fully-extended configuration.

FIG. 7 is a side sectional view illustrating an alternative embodiment of the distal portion of a lead extraction device.

FIG. 8 is a side sectional view illustrating an alternative embodiment of the distal portion of a lead extraction device.

### DETAILED DESCRIPTION

The following description is exemplary in nature and is not intended to limit the scope, applicability, or configuration of the present disclosure in any way. Rather, the description provides practical illustrations for implementing exemplary embodiments of the present disclosure.

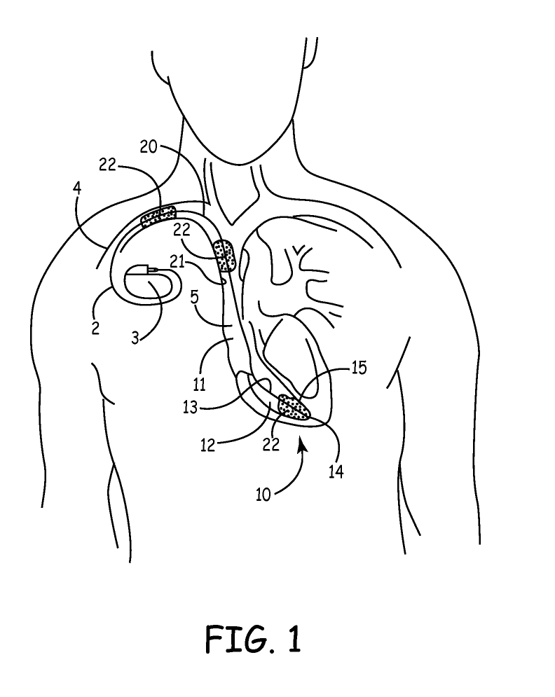

To better understand the environment in which lead 2 exists and from which it is to be removed, FIG. 1 shows a diagrammatic view of a heart 10 with the implanted lead 2. As seen in FIG. 1, a lead 2 connects a pacemaker 3 to heart 10 through the right subclavian vein 4, the superior vena cava 5 and down into the heart 10. While not intended to be limiting, lead 2 is shown specifically in the right ventricle 12; the location of lead 2 may also be in the right atrium 11. Distal end 13 of lead 2 includes an electrode 14 for electrically stimulating the heart 10 and a plurality of tines 15 to provide fixation of lead 2 within heart 10. During chronic implantation, lead 2 becomes affixed along its side surfaces 20 to inner surfaces 21 of the venous system and at its distal end 13 to heart 10 through the formation of fibrous scar tissue 22. It is therefore desirable to separate such fibrous tissue 22 from lead 2 during removal of the lead 2.

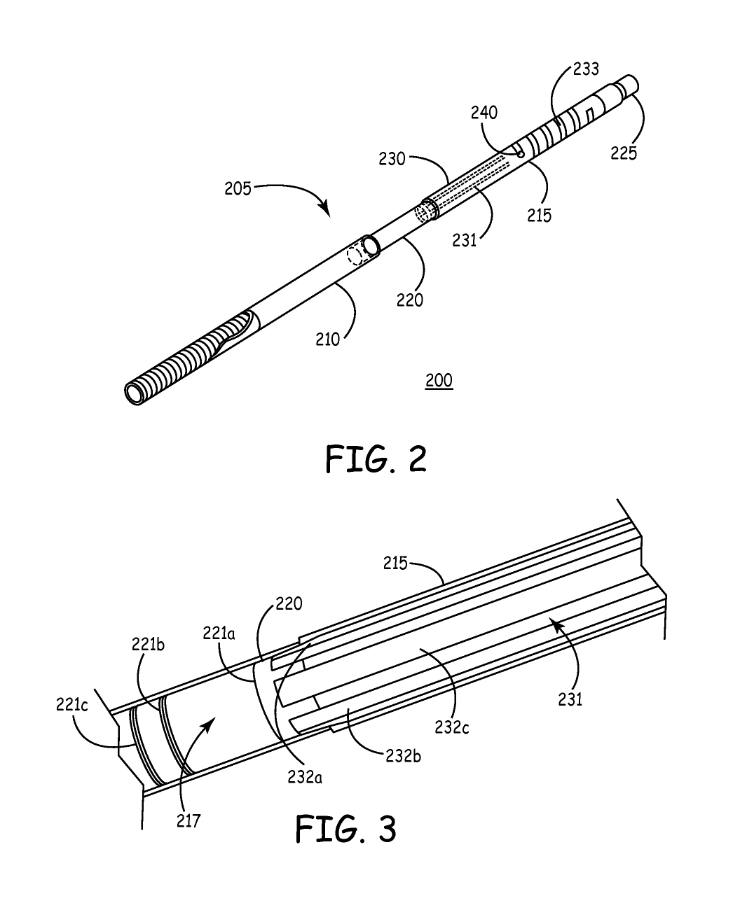

Turning to FIG. 2, a side sectional view of a lead extraction device 200 in accordance with an embodiment of the present disclosure is illustrated. Lead extraction device 200 may include a shaft 210 that is coupled to an outer tube 215. In an embodiment, shaft 210 and outer tube 215 may be formed as a unitary piece.

Alternatively, shaft 210 and outer tube 215 may be formed separately and coupled together in an end to end configuration. A restrictor tube 220 may additionally be coupled between outer tube 215 and shaft 210 in an end to end configuration to form a single continuous elongate body 205. The coupling between shaft 210 to restrictor tube 220 and restrictor tube 220 to outer tube 215 may be performed in any suitable bonding manner known in the art, including but not limited, to an adhesive material or a frictional fit. Whether the restrictor tube 215 is present or not, it should be understood that body 205 of the lead extraction device 200 is defined by the length from the proximal end of the shaft 210 to the distal end of the outer tube 215.

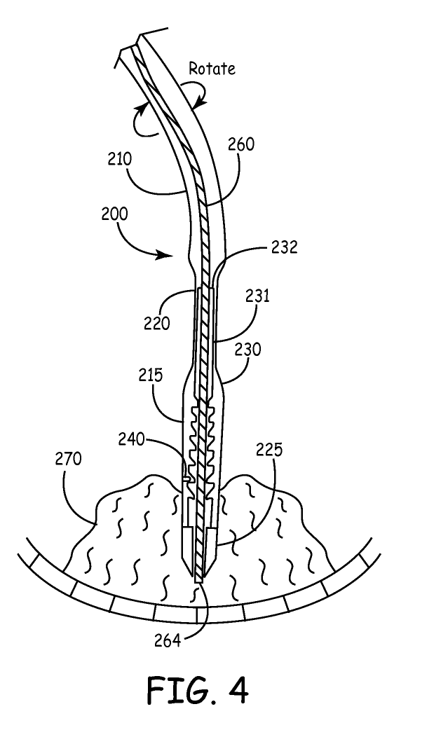

In use, lead extraction device 200 tracks over the lead (FIG. 4) to be extracted. Body 205 defines a lumen 217 (FIG. 3) that is configured to permit lead extraction device 200 to be introduced over the lead and tracked over the lead. Specifically, lumen 217 extends from a proximal end of the braided shaft to the distal end outer tube 215.

A coring tip 225 is coupled to the distal end of outer tube 215. Coring tip 225 may have sharp outward serrations or ridges for separating tissue from the lead to be extracted. It may be desirable to provide an outward taper on the interior surface of the distal leading edge of coring tip 225. The taper will prevent contact between the sharp coring tip 225 and the lead to be extracted which may cause an incision of the lead. Coring tip 225 may be coupled to the outer tube 215 in an end-to-end manner. Alternatively or in addition, a frictional fit may be used whereby a portion of coring tube 225 may be embedded in the distal opening of outer tube 215. Coring tip 225 may be coupled using any bonding technique known in the art that is suitable for bonding the materials.

The outer tuber 215 may include an engagement mechanism such as an inner tubular member 230 that has a first portion for selectively engaging the lead to be extracted and a second portion for threadedly engaging the outer tube 215. Inner tubular member 230 may alternatively be disposed in a sliding relationship with the outer tube 215. In the illustrated embodiment, inner tubular member 230 includes a threaded portion 233 that rotates around a coupling pin 240. Inner tubular member 230 may extend approximately within the entire length of outer tube 215. Coupling pin 240 is bonded to outer tuber 215 with at least a portion of coupling pin 240 extending within inner lumen 217 to engage the threaded portion 233. The coupling between inner tubular member 230 to coupling pin 240 permits rotation and axial movement of the outer tube 215.

Inner tubular member 230 may include a slotted portion 231. Slotted portion 231 may have one or more slits extending axially along the length of inner tubular member 230 to define one or more fingers 232 (a-c) (FIG. 3). Alternatively, slotted portion 231 may be formed as a plurality of curvilinear segments defining an alternating circumferential pattern (discussed in FIG. 8). Whether implemented as a slotted tube or as a plurality of curvilinear segments, slotted portion 231 facilitates radial expansion or contraction of at least a portion of the inner tubular member 230. The contraction of slotted portion 231 permits the inner tubular member 230 to engage to engage the lead (not shown) and thereby remain in a fixed position or have restricted movement.

In operation, outer tube 215 rotates around inner tubular member 230 to advance and retreat longitudinally over a lead (not shown) that is to be extracted. The rotation of outer tube 215 around inner tubular member 230 is permitted by the static position or restricted movement of the inner tubular member 230. In other words, the engagement of coupling pin 240 to inner tubular member 230 permits both rotational and axial movement of inner tubular member 230 in relation to inner lumen 217.

Lead extraction device 200 may be dimensioned to have an inner lumen 217 that fits over the outer body of an implanted lead with a clearance provided between inner lumen 217 to facilitate uninhibited movement of device 200 over the lead body. Lead extraction device 200 may preferably be constructed from biocompatible materials. The inner tubular member 230 and restrictor tube 220 may be made from a nickel-titanium alloy material such as Nitinol, stainless steel, titanium, tantalum, and other metals and metal alloys. Coring tip 225 and coupling pin 240 may be made of stainless steel. Shaft 210 may be formed as a braided shaft having a plurality of layers fused together. Forming shaft 210 as a braided shaft may be desirable as it permits sufficient torque to be applied to the shaft 210 without resulting in kinking of the lead extraction device 200. An inner layer of shaft 210 is preferably made of a material having a low friction coefficient such as polytetrafluoroethylene (PTFE) or a blended nylon. A middle layer of shaft 210 may be made of a braided wire, such as stainless steel or copper. The middle layer is covered by a plastic material such as polyaryletheretherketone (PEEK) thermoplastic, PARYLENE® polyxylylene polymers, or a suitable polymer material. The outer tube 215 may also be made of a plastic material.

FIG. 3 depicts a sectional view of a distal portion of one embodiment of lead extraction device 200. The illustration depicts slotted portion 231 disposed partially within outer tube 215 and partially within restrictor tube 220. The slits in slotted portion 231 extend axially to form fingers 232 (a-c). It should be noted that the number or orientation of the slits in the slotted portion 231 is predicated on permitting the contraction of the diameter of the inner tubular member 230 as desired so as to grip the lead that is to be extracted. As such, the disclosure is not limited to formation of slotted regions and it is contemplated that one skilled in the art could formulate a suitable substitute without undue experimentation. The proximal end of slotted portion 231 may be chamfered at any suitable angle to facilitate the insertion of slotted portion 231 into restrictor tuber 220.

In an embodiment, the inner diameter of the lumen 217 in the restrictor tube 220 may be narrowed from the distal end to the proximal end. The narrowing of the inner diameter of restrictor 220 permits the contraction of the diameter of slotted portion 231 during the axial movement of inner tubular member 230 toward the proximal end of inner lumen 217. The contraction of the diameter of slotted portion 231 facilitates the engagement of inner tubular member 230 with the lead to be extracted. Narrowing of the inner diameter of restrictor 220 may be achieved through the use of ribs 221 (a-c). Ribs 221 (a-c) may have successively graduated thickness to permit successively-increased radial contraction of the slotted portion 231.

FIG. 4 depicts the coupling of a distal portion of the lead extraction device 200 with an implanted lead 260 as the lead extraction device 200 would be used to remove the lead 260. The coring tip 225 of lead extraction device 200 is introduced over a proximal end (not shown) of a lead 260 to be extracted. The lead extraction device 200 is tracked over lead 260 and advanced toward the distal end 264 of lead 260 that is embedded in tissue 270. The coring tip 225 encounters an obstruction when the lead extraction device 200 is successfully advanced to contact the tissue 270.

The shaft 210 may then be rotated and the rotation of shaft 210 translates into rotation of outer tube 215. As previously described, because of the threaded engagement between coupling pin 240 with inner tubular member 230, the rotation of outer tube 215 will result in concurrent axial movement of the outer tube 215. One consequence of rotation of outer tube 215 is that the fingers 232 (a-c) are advanced into the restrictor tube 220. As the fingers 232 (a-c) advance into the restrictor tube 220, the radial dimension of the slotted portion 231 is contracted. The reduced radial dimension of the slotted portion 231 will permit the fingers 232 (a-c) to anchor onto the lead 260. Additionally, as the outer tube 215 is rotated, the coring tip 225 advances into the tissue 270. The advancement of coring tip 225 into tissue 270 creates separation of lead 260 from the tissue 270 which permits the lead 260 to be withdrawn. In addition, shaft 210 may be rotated in the opposing direction to release the grip by fingers 232 (a-c) of lead 260. Releasing the grip permits the coupling device 200 to further be tracked along the lead 260 for separation of tissue that may be located in further distally on the lead 260. The gripping, coring, release and regripping of the lead 260 may be repeated, as necessary, until all the tissue surrounding the lead 260 is successfully released.

Once the tissue surrounding the lead 260 is released from fibrous tissue 270, only fibrous tissue proximate the distal face of lead 270 retains lead 270. At this point traction may be applied to lead 270 by tugging at lead extraction device 200. The engagement of slotted portion 231 may be beneficial as it secures the lead 270 at a location proximate the region where the lead is retained by fibrous tissue.

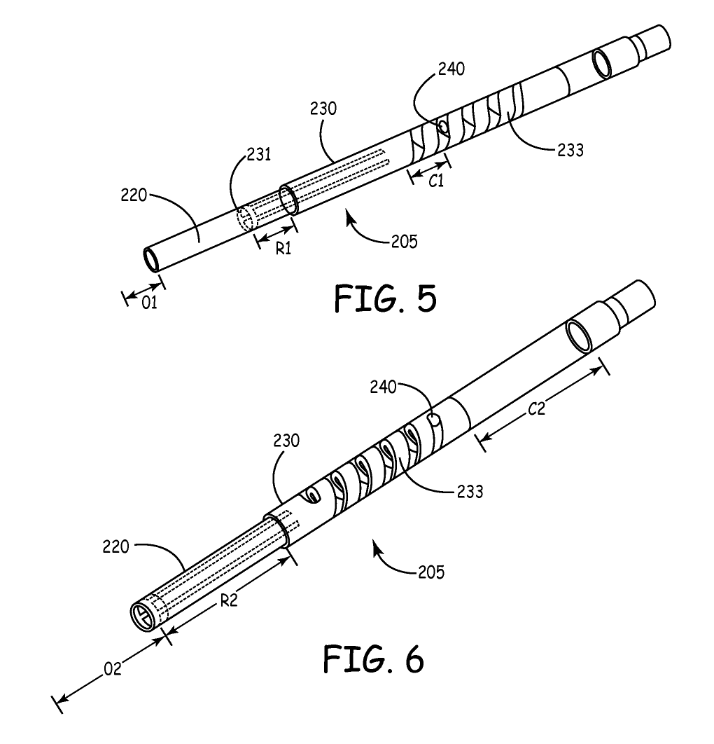

FIG. 5 is a side sectional view of the lead extraction device 200 of FIG. 2 showing the device 200 in an open configuration. The open configuration is, generally, any diameter of the slotted portion 231 that permits the lead extraction device 200 to be tracked over the body of the lead to be extracted. In the embodiment, slotted portion 231 is illustrated as being partially-extended into the restrictor tube 220. Rotational movement of body 205 with respect to inner tubular member 230 has caused axial movement to a length O1 that is equivalent to a length C1, which is the length that the threaded portion 233 has moved with respect to the coupling pin 240. The slotted portion 231 is depicted as having been advanced within the restrictor tube 220 to a length R1 that is also equivalent to length C1. As described above with respect to FIG. 3, the narrowing of the diameter of inner lumen 217 in the region of the restrictor tube from the distal end to the proximal end constricts the diameter of slotted portion 231. This constriction in the diameter will occur generally in the region of length R1 and as such the lead to be extracted will be engaged at the general location of R1.

FIG. 6 is a side sectional view of the lead extraction device 200 of FIG. 2 showing the device 200 in a closed configuration. In contrast to the open configuration, the diameter of slotted portion 231 in the closed configuration will engage and grip the body of the lead to be extracted. The gripping by slotted portion 231 may facilitate the rotational and axial movement of the coring tip 225 without corresponding movement of the lead to be extracted. The illustration shows the slotted portion 231 having been advanced within the restrictor tube 220 to a length R2 that is equivalent to a length C2. Length C2 represents the axial length to which the threaded portion 233 has moved with respect to the coupling pin 240. Slotted portion 231 has advanced within the restrictor tube 220 to a length R2 that is equivalent to length C2. Consequently, the net movement of body 205 with respect to the inner tubular member 230 is equivalent to the length C2, represented by the dimension O2.

Referring to both FIG. 5 and FIG. 6, it should be understood that the coring tip 225 will be advanced along with the rest of body 205 due to the fixed relation of coring tip 225 to body 205. Therefore, during operation of the lead extraction device 200, the length C1 and C2 exemplify the length over which the coring tip 225 will be advanced over the lead, as it separates tissue surrounding the lead from the lead. The actual length to which the coring tip 225 (in conjunction with body 205) is advanced depends on the length to which the lead is embedded into the tissue. As such, the actual physical lengths of the various components of lead extraction device 200 are a matter of design choice and these lengths are not critical to the interrelation of the components.

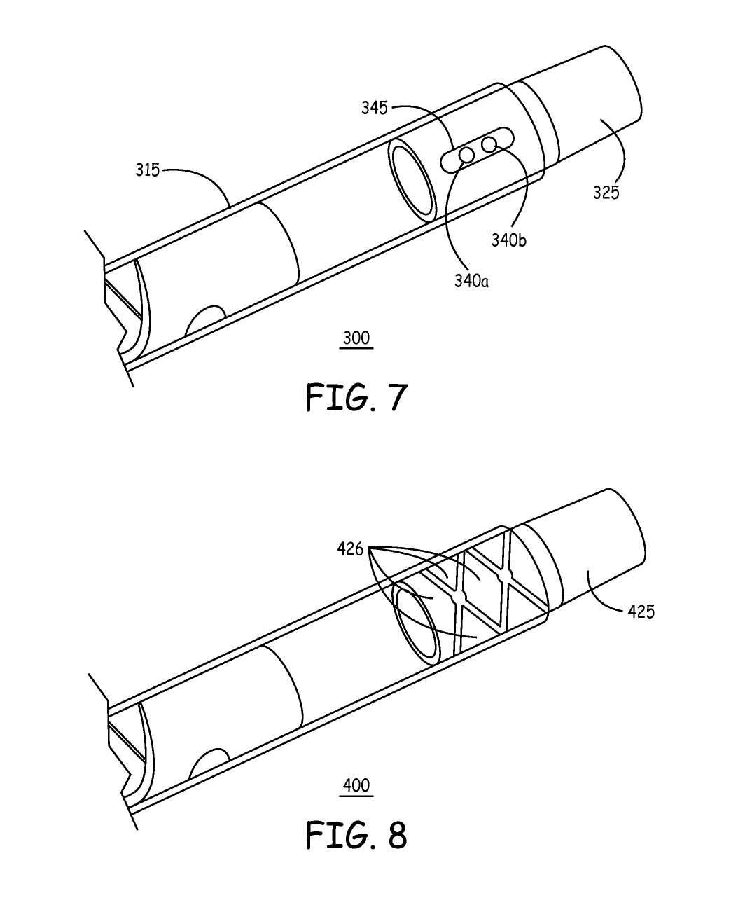

Turning now to FIG. 7, a side sectional view of an alternative embodiment of the distal portion of a lead extraction device 300 is illustrated. A coring tip 325 is coupled to an outer tube 315 with a plurality of coupling pins 340 (a-b) mounted thereon. It should be noted that the depiction of the number and location of coupling pins 340 (a-b) is merely provided for ease of illustration. In an actual implementation of the embodiment, any number or orientation of coupling pins 340 (a-b) may be utilized without departing from the spirit and scope of the illustrative embodiment.

In use, the coupling pins 340 (a-b) permit movement of the coring tip 325 axially over the length of a slot 345 that is provided in the wall of outer tube 315. As the coring tip 325 is advanced over the lead to be extracted, the tip 325 will come in contact with the tissue covering the lead. Further advancement of the lead extraction device 300 will cause axial movement of the coring tip 325 in the direction from the distal end to the proximal end of the lead extraction device 300. Coupling pin 340a engages with the end of slot 345 to prevent further movement of coring tip 325.

The sliding-engagement of coring tip 325 within the slot 345 of outer tube 315 may be utilized to cause axial movement of an inner tubular member 330 (similar to inner tubular member 230 discussed in relation to FIG. 3) in a direction from the distal end to the proximal end of lead extraction device 300. This movement of inner tubular member 330 may permit the member 330 to engage the lead that is to be extracted.

FIG. 8 illustrates an alternative embodiment of the distal portion of the lead extraction device 400. A coring tip 425 is illustrated as having a plurality of radially spaced apart curvilinear segments 426 defining an alternating circumferential pattern and a longitudinal axis. The spaced apart segments 426 permit radial expansion of the cylindrically-shaped coring tip 425 from a contracted state to an expanded state. Radial expansion of the spaced apart segments 426 will prevent the coring tip 425 from gripping the lead that is to be extracted. This in turn facilitates forward, axial movement of the lead extraction device 400. Coring tip 425 may be constructed through any suitable construction method such as the stent construction method described in U.S. Pat. No. 6,863,684 issued to Steven W. Kim et al., incorporated herein by reference in its entirety.

In the foregoing detailed description, the present disclosure has been described in considerable detail in order to comply with the patent statutes and to provide those skilled in the art with specific implementations that facilitate the understanding of the novel principles of the disclosure. However, it is to be understood that the principles of the present disclosure can be carried out by specifically different equipment and devices and that various modifications, both as to the equipment and operating procedures, can be accomplished without departing from the scope of the disclosure as set forth in the appended claims.

What is claimed is:

1. A lead extraction device for extraction of a lead comprising:
an elongate body having an inner lumen with a radial dimension configured to fit over the lead, including:
a tubular member;
an engagement mechanism disposed within the tubular member, wherein a first portion of the engagement mechanism is selectively engageable with the lead; and
a coring tip coupled to the elongate body in an end to end configuration.

2. The lead extraction device of claim 1, further comprising a coupling pin coupled to the tubular member configured for threaded engagement with a second portion of the engagement mechanism.

3. The lead extraction device of claim 1, wherein the tubular member comprises:
a braided shaft; and
an outer tube coupled to the braided shaft, wherein the engagement mechanism is disposed within the outer tube.

4. The lead extraction device of claim 1, wherein in a first configuration the engagement mechanism has a first radial dimension that permits longitudinal movement of the engagement mechanism over the lead; and
wherein in a second configuration the engagement mechanism has a second radial dimension that is smaller that the first radial dimension and grips the lead to prevent longitudinal movement.

5. The lead extraction device of claim 4, further comprising a restrictor tube wherein the engagement mechanism is partially disposed within the restrictor tube in the second configuration.

6. The lead extraction device of claim 5, wherein the restrictor tube includes a plurality of ribs.

7. The lead extraction device of claim 1 wherein the engagement mechanism comprises:
a threaded portion; and
a slotted region having a plurality of slits along a longitudinal axis, wherein the slits permit compression of a radial dimension of the engagement mechanism.

8. The lead extraction device of claim 1 wherein the engagement mechanism comprises:
a threaded portion; and
a plurality of radially spaced apart segments defining a circumference and a longitudinal axis coupled to the threaded portion, wherein the radially spaced apart segments permit compression of a radial dimension of the engagement mechanism.

9. The lead extraction device of claim 1 wherein the coring tip includes a plurality of radially spaced apart segments defining a circumference and a longitudinal axis, wherein the radially spaced apart segments permit expansion of a radial dimension of the coring tip.

10. The lead extraction device of claim 1 wherein the tubular member is fabricated from at least one material selected from the group consisting of stainless steel, nitinol, titanium, tantalum, and other metals and metal alloys.

11. The lead extraction device of claim 1 wherein the tubular member is formed as a unitary piece.
